(19) United States
(12) Patent Application Publication
(10) Pub. No.: US 2005/0182122 A1
(43) Pub. Date: Aug. 18, 2005

(54) Method of treating abnormal cell growth using indolinone compounds

(76) Inventors: Carlo Leonel Bello, San Francisco, CA (US); Stephen Kelsey, Montara, CA (US); Giorgio Pietro Massimini, Abbiategrasso (IT); Shem J. Patyna, Encinitas, CA (US); Paul Scigalla, Berlin (DE)

Correspondence Address: AGOURON PHARMACEUTICALS, INC., 10777 SCIENCE CENTER DRIVE, SAN DIEGO, CA, 92121, US

(21) Appl. No.: 10/991,244

(22) Filed: Nov. 17, 2004

Related U.S. Application Data
(60) Provisional application No. 60/524,133, filed on Nov. 20, 2003.

(51) Int. Cl. A61K 31/405

(52) U.S. Cl. 514/414

(57) ABSTRACT

The invention provides a method of treating abnormal cell growth in a mammal, such as a human, by administering to the mammal a therapeutically effective amount of a composition including one or both of 5-(5-fluoro-2-oxo-1,2-dihydroindol-(3Z)-ylidenemethyl)-2,4-dimethyl-1H-pyrrole-3-carboxylic acid (2-diethylaminoethyl)-amide and 5-(5-fluoro-2-oxo-1,2-dihydroindol-(3Z)-ylidenemethyl)-2,4-dimethyl-1H-pyrrole-3-carboxylic acid (2-ethylaminoethyl)-amide, or pharmaceutically acceptable salts, solvates or hydrates thereof, for at least one cycle of an intermittent dosing regimen.

This application claims the benefit of U.S. Provisional Application No. 60/524,133, filed Nov. 20, 2003, the disclosure of which is incorporated herein by reference in its entirety.

### BACKGROUND OF THE INVENTION

This invention relates to methods of treatment of abnormal cell growth, such as cancer, in mammals. In particular, the invention provides methods of treatment of abnormal cell growth using indolinone derivatives that inhibit multiple receptor tyrosine kinases.

The compound 5-(5-fluoro-2-oxo-1,2-dihydroindol-(3Z)-ylidenemethyl)-2,4-dimethyl-1H-pyrrole-3-carboxylic acid (2-diethylaminoethyl)-amide, represented by formula 1 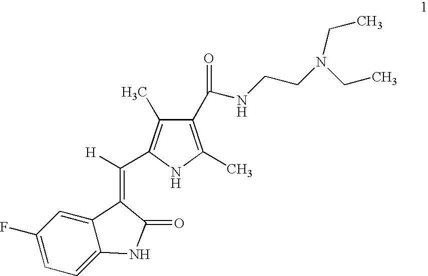
is an oral, novel cancer drug in the early stages of clinical development. Compound 1 targets multiple receptor tyrosine kinase inhibitors, including PDGFR, KIT and VEGFR, combining multiple targets and mechanisms to fight abnormal cell growth, such as cancer, in a single compound. Compound 1, also referred to in the literature as “SU11248 or SU011248”, is described more fully in a variety of literature references, such as: Abrams, T. J., Lee, L. B., Murray, L. J., Pryer, N. K. and Cherrington, J. M., “SU11248 inhibits KIT and PDGFRβ in preclinical models of human small cell lung cancer,” Molecular Cancer Therapeutics 2:471-478 (2003); O'Farrell, A. M., Abrams, T. J., Yuen, H., Ngai, T. J., Yee, K., Louie, S. G., Wong, L. M., Hong, W., Lee, L. B., Town, A., Smolich, B. D., Manning, W. C., Murray, L. J., Heinrich, M. and Cherrington, J. M., “SU11248 is a novel FLT3 tyrosine kinase inhibitor with potent FLT3 activity in vitro and in vivo,” Blood 101:3597-3605 (2003); Mendel, D. B., Laird, A. D., Xin, X., Louie, S. G., Christensen, J. G., Li, G., Schreck, R. E., Abrams, T. J., Ngai, T. J., Lee, L. B., Murray, L. J., Carver, J., Chan, E., Moss, K. G., Haznedar, J. Ö., Sukbuntherng, J., Blake, R. A., Sun, C., Tang, C., Miller, T., Shirazian, S., McMahon, G. and Cherrington, J. M., “In vivo anti-tumor activity of SU11248, a novel tyrosine kinase inhibitor targeting vascular endothelial growth factor and platelet-derived growth factor receptors: determination of a pharmacokinetic/pharmacodynamic relationship,” Clinical Cancer Res. 9:327-337 (2003); and Sun, L., Liang, C., Shirazian, S., Zhou, Y., Miller, T., Cui, J., Fukuda J. Y., Chu, J.-Y., Nematalla, A., Wang, X., Chen, H., Sistla, A., Luu, T. C., Tang, F., Wei, J. and Tang C., “Discovery of 5-[5-Fluoro-2-oxo-1,2-dihydroindol-(3Z)-ylidenemethyl]-2,4-dimethyl-1H-pyrrole-3-carboxylic Acid (2-Diethylaminoethyl)amide, a Novel Tyrosine Kinase Inhibitor Targeting Vascular Endothelial and Platelet-Derived Growth Factor Receptor Tyrosine Kinase,” J. Med. Chem. 46, 1116-1119. (2003). The disclosures of these references are incorporated herein by reference in their entireties.

The compound of formula 1 has several known metabolites, including the compounds of formulae 2, 3 and 4. 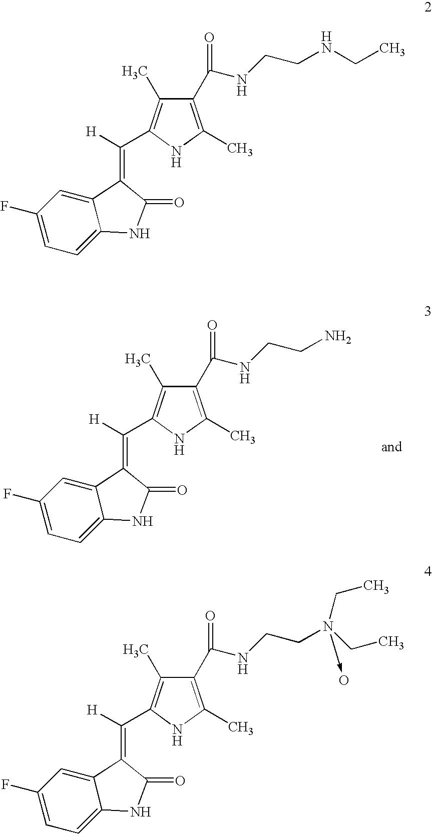
Of these metabolites, the compound of formula 2,5-(5-fluoro-2-oxo-1,2-dihydroindol-(3Z)-ylidenemethyl)-2,4-dimethyl-1H-pyrrole-3-carboxylic acid (2-ethylaminoethyl)-amide, shows similar multi-receptor tyrosine kinase inhibitor activity as compound 1.

Administration of the compound of formula 1, or its active metabolite 2, in an effective dosing regimen, must take into account the long blood/plasma half lives of these two compounds, approximately 40 and 80 hours, respectively, as well as their safety profiles. Thus, there is a need for safe and therapeutically effective dosing regimens for administering the compounds of formula 1 and 2 to a mammal, including a human, for treating abnormal cell growth, such as cancer.

### SUMMARY OF THE INVENTION

In one embodiment, the invention provides a method of treating abnormal cell growth in a mammal, such as a human, by administering to the mammal a therapeutically effective amount of a composition comprising at least one of 5-(5-fluoro-2-oxo-1,2-dihydroindol-(3Z)-ylidenemethyl)-2,4-dimethyl-1H-pyrrole-3-carboxylic acid (2-diethylaminoethyl)-amide or 5-(5-fluoro-2-oxo-1,2-dihydroindol-(3Z)-ylidenemethyl)-2,4-dimethyl-1H-pyrrole-3-carboxylic acid (2-ethylaminoethyl)-amide, or pharmaceutically acceptable salts, solvates or hydrates thereof, for at least one cycle of an intermittent dosing regimen.

In a particular aspect of this embodiment, a cycle of the intermittent dosing regimen comprises a treatment period and a rest period, and the intermittent dosing regimen comprises: (a) administering the composition in the treatment period in an amount sufficient to provide a Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of from 10 to 220 ng/mL; and (b) discontinuing administration of the composition in the rest period for a duration sufficient to achieve a blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of less than 10 ng/mL.

In preferred aspects of this embodiment, the Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide in the treatment period is in a range from a lower limit of 10 or 30 or 50 or 75 ng/mL, to an upper limit of 220 or 200 or 150 or 125 or 100 ng/mL, with ranges from any lower limit to any upper limit being contemplated. Specific examples of particularly preferred ranges include, but are not limited to, 10 to 220 ng/mL, 30 to 150 ng/mL, and 50 to 100 ng/mL.

In a preferred aspect of this embodiment, the blood/plasma concentration in the rest period is less than 5 ng/mL, preferably less than 1 ng/mL.

In another preferred aspect of this embodiment, the therapeutically effective amount is in a range of from a lower limit of 10 or 20 or 30 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, to an upper limit of 150 or 125 or 100 or 80 or 75 or 70 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, with ranges from any lower limit to any upper limit being contemplated. Specific examples of preferred ranges include, but are not limited to, 10 to 100 mg, 20 to 80 mg and 30 to 75 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide. In a further preferred aspect, the composition is provided in tablets or capsules containing 12.5 or 25 mg of the active pharmaceutical ingredient, expressed as free base equivalent of 5-(5-fluoro-2-oxo-1,2-dihydroindol-(3Z)-ylidenemethyl)-2,4-dimethyl-1H-pyrrole-3-carboxylic acid (2-diethylaminoethyl)-amide. In this aspect, specific examples of preferred dosages include, but are not limited to, 12.5, 25, 37.5, 50, 62.5, 75, 87.5, 100, 112.5, 125, 137.5 and 150 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

In particular aspects of this embodiment, the composition is administered for at least 2 cycles, at least 3 cycles, at least 4 cycles, at least 5 cycles, or at least 6 cycles of the intermittent dosing regimen.

In particular aspects of this embodiment, the composition is administered at least once per day, or at least once per two days, or at least once per three days, during the treatment period.

In a particular aspect of this embodiment, the treatment period has a duration of at least 7 days, or at least 10 days, or at least 14 days, or at least 21 days, or at least 28 days.

In a particular aspect of this embodiment, the rest period has a duration of at least 3 days, or at least 5 days, or at least 7 days, or at least 10 days, or at least 14 days.

It should be appreciated that any combination of the recited treatment period and rest period durations is contemplated. Specific examples of combinations include, but are not limited to, a treatment period of at least 14 days and a rest period of at least 7 days; a treatment period of at least 14 days and a rest period of at least 14 days; a treatment period of at least 21 days and a rest period of at least 7 days; a treatment period of at least 28 days and a rest period of at least 7 days; and a treatment period of at least 28 days and a rest period of at least 14 days.

In another embodiment, the invention provides a method of treating cancer in a mammal, the method comprising: (a) administering to the mammal in a treatment period, a therapeutically effective amount of a composition comprising at least one of 5-(5-fluoro-2-oxo-1,2-dihydroindol-(3Z)-ylidenemethyl)-2,4-dimethyl-1H-pyrrole-3-carboxylic acid (2-diethylaminoethyl)-amide or 5-(5-fluoro-2-oxo-1,2-dihydroindol-(3Z)-ylidenemethyl)-2,4-dimethyl-1H-pyrrole-3-carboxylic acid (2-ethylaminoethyl)-amide, or pharmaceutically acceptable salts, solvates or hydrates thereof, in an amount sufficient to provide a Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of from 10 to 220 ng/mL; (b) discontinuing administration of the composition in a rest period, for a duration sufficient to achieve a blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of less than 10 ng/mL; and (c) repeating steps (a) and (b).

In preferred aspects of this embodiment, the Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide in the treatment period is in a range from a lower limit of 10 or 30 or 50 or 75 ng/mL, to an upper limit of 220 or 200 or 150 or 125 or 100 ng/mL, with ranges from any lower limit to any upper limit being contemplated. Specific examples of particularly preferred ranges include, but are not limited to, 10 to 220 ng/mL, 30 to 150 ng/mL, and 50 to 100 ng/mL.

In a preferred aspect of this embodiment, the blood/plasma concentration in the rest period is less than 5 ng/mL, preferably less than 1 ng/mL.

In another preferred aspect of this embodiment, the therapeutically effective amount is in a range of from a lower limit of 10 or 20 or 30 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, to an upper limit of 150 or 125 or 100 or 80 or 75 or 70 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, with ranges from any lower limit to any upper limit being contemplated. Specific examples of preferred ranges include, but are not limited to, 10 to 100 mg, 20 to 80 mg and 30 to 75 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide. In a further preferred aspect, the composition is provided in tablets or capsules containing 12.5 or 25 mg of the active pharmaceutical ingredient, expressed as free base equivalent of 5-(5-fluoro-2-oxo-1,2-dihydroindol-(3Z)-ylidenemethyl)-2,4-dimethyl-1H-pyrrole-3-carboxylic acid (2-diethylaminoethyl)-amide. In this aspect, specific examples of preferred dosages include, but are not limited to, 12.5, 25, 37.5, 50, 62.5, 75, 87.5, 100, 112.5, 125, 137.5 and 150 mg per day, expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

In particular aspects of this embodiment, the composition is administered at least once per day, or at least once per two days, or at least once per three days, during the treatment period.

In a particular aspect of this embodiment, the treatment period has a duration of at least 7 days, or at least 10 days, or at least 14 days, or at least 21 days, or at least 28 days.

In a particular aspect of this embodiment, the rest period has a duration of at least 3 days, or at least 5 days, or at least 7 days, or at least 10 days, or at least 14 days.

It should be appreciated that any combination of the recited treatment period and rest period durations is contemplated. Specific examples of combinations include, but are not limited to, a treatment period of at least 14 days and a rest period of at least 7 days; a treatment period of at least 14 days and a rest period of at least 7 days; a treatment period of at least 21 days and a rest period of at least 7 days; a treatment period of at least 28 days and a rest period of at least 7 days; and a treatment period of at least 28 days and a rest period of at least 14 days.

In preferred aspects of this embodiment, steps (a) and (b) are repeated at least twice, i.e., a total of at least two (a)-(b) cycles, or at least 3 times, or at least 4 times, or at least 5 times, or at least 6 times.

In a specific embodiment of any of the inventive methods described herein, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In a particular aspect of this embodiment, the cancer is selected from gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, and combinations thereof.

In further specific embodiments of any of the inventive methods described herein, the method further comprises administering to the mammal an amount of one or more substances selected from anti-tumor agents, anti-angiogenesis agents, signal transduction inhibitors, and antiproliferative agents, which amounts are together effective in treating said abnormal cell growth. Such substances include those disclosed in PCT publication nos. WO 00/38715, WO 00/38716, WO 00/38717, WO 00/38718, WO 00/38719, WO 00/38730, WO 00/38665, WO 00/37107 and WO 00/38786, the disclosures of which are incorporated herein by reference in their entireties.

Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as taxol (paclitaxel), docetaxel (Taxotere), 2′-N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethylnitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2,4-diamino-5-methyl-6-quinazolinyl)methyl]amino]benzoyl]-L-aspartic acid, N-[4-[[(2,4-diamino-5-ethyl-6-quinazolinyl)methyl]amino]benzoyl]-L-aspartic acid, N-[2-chloro-4-[[(2,4-diaminopteridinyl)methyl]amino]benzoyl]-L-aspartic acid, soluble Bakers antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred ant-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloprotienase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Examples of MMP inhibitors include AG-3340, RO 32-3555, RS 13-0830, and the compounds recited in the following list:
3-[[4-(4-fluoro-phenoxy)-benzenesulfonyl]-(1-hydroxycarbamoyl-cyclopentyl)-amino]-propionic acid;
3-exo-3-[4-(4-fluoro-phenoxy)-benzenesulfonylamino]-8-oxa-bicyclo[3.2.1]octane-3-carboxylic acid hydroxyamide;
(2R, 3R) 1-[4-(2-chloro-4-fluoro-benzyloxy)-benzenesulfonyl]-3-hydroxy-3-methyl-piperidine-2-carboxylic acid hydroxyamide;
4-[4-(4-fluoro-phenoxy)-benzenesulfonylamino]-tetrahydro-pyran-4-carboxylic acid hydroxyamide;
3-[[4-(4-fluoro-phenoxy)-benzenesulfonyl]-(1-hydroxycarbamoyl-cyclobutyl)-amino]-propionic acid;
4-[4-(4-chloro-phenoxy)-benzenesulfonylamino]-tetrahydro-pyran-4-carboxylic acid hydroxyamide;
3-[4-(4-chloro-phenoxy)-benzenesulfonylamino]-tetrahydro-pyran-3-carboxylic acid hydroxyamide;
(2R, 3R) 1-[4-(4-fluoro-2-methyl-benzyloxy)-benzenesulfonyl]-3-hydroxy-3-methyl-piperidine-2-carboxylic acid hydroxyamide;
3-[[4-(4-fluoro-phenoxy)-benzenesulfonyl]-(1-hydroxycarbamoyl-1-methyl-ethyl)-amino]-propionic acid;
3-[[4-(4-fluoro-phenoxy)-benzenesulfonyl]-(4-hydroxycarbamoyl-tetrahydro-pyran-4-yl)-amino]-propionic acid;
3-exo-3-[4-(4-chloro-phenoxy)-benzenesulfonylamino]-8-oxa-bicyclo[3.2.1]octane-3-carboxylic acid hydroxyamide;
3-endo-3-[4-(4-fluoro-phenoxy)-benzenesulfonylamino]-8-oxa-bicyclo[3.2.1]octane-3-carboxylic acid hydroxyamide; and
3-[4-(4-fluoro-phenoxy)-benzenesulfonylamino]-tetrahydro-furan-3-carboxylic acid hydroxyamide;
   and pharmaceutically acceptable salts, solvates and prodrugs of said compounds.

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example AG-13736 (Pfizer, Inc.), can also be combined or co-administered with the composition. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,534,524, WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); Avastin™ or bevacizumab, an anti-VEGF monoclonal antibody (Genentech, Inc. of South San Francisco, Calif.); and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with the composition. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following United States patent applications: Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

The composition may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

Specific examples of combination therapy can be found in PCT Publication No. WO 03/015608 and U.S. Patent Publication No. 2004-0152759, the disclosures of which are incorporated herein by reference in their entireties.

Definitions

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bistosylate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Particularly preferred salts include L-malate salts.

The term “prodrug”, as used herein, unless otherwise indicated, means compounds that are drug precursors, which following administration, release the drug in vivo via some chemical or physiological process (e.g., a prodrug on being brought to the physiological pH is converted to the desired drug form).

The invention also includes isotopically-labeled compounds, which are identical to those recited in Formula 1 or 2, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula 1 or 2 of this invention and prodrugs thereof can generally be prepared by carrying out the procedures described for the non-labeled compound, substituting a readily available isotopically labeled reagent for a non-isotopically labeled reagent.

### BRIEF DESCRIPTION OF THE DRAWINGS

FIG. 1 shows minimum blood/plasma concentrations Cₘᵢₙ for 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide for several patients, according to Example 2.

FIG. 2 shows minimum blood/plasma concentrations Cₘᵢₙ for 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide for several patients, according to Example 3.

### DETAILED DESCRIPTION OF THE INVENTION

Compounds of formula 1 and 2, and salts thereof, can be prepared as described in U.S. Pat. No. 6,573,293; PCT publication Nos. WO 01/60814, WO/03/016305 and WO 03/070725; U.S. Patent Application Publication No. 2003/0069298; U.S. Provisional Patent Application No. 60/501,994, filed Sep. 11, 2003; and R. Vaidyanathan et al., “Early amidation approach to 3-[(4-amido)pyrrole-2-yl]-2-indolinones,” Journal of Organic Chemistry, 68, 6447-6450 (2003), the disclosures of which are incorporated herein by reference in their entireties. Certain starting materials may be prepared according to methods familiar to those skilled in the art and certain synthetic modifications may be done according to methods familiar to those skilled in the art.

The compounds of formula 1 and 2 are capable of forming a wide variety of different salts with various inorganic and organic acids. Although such salts must be pharmaceutically acceptable for administration to mammals, it is often desirable in practice to initially isolate the compound of formula 1 or 2 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid. Specific examples of preparation of a preferred salt, the L-malate salt, can be found in U.S. Patent Application Publication No. 2003/0069298 and WO/03/016305, the disclosures of which are incorporated herein by reference in their entireties.

Administration of the compound of formula 1 or 2 can be effected by any method that enables delivery of the compound to the site of action. These methods include oral routes, intraduodenal routes, parenteral injection (including intravenous, subcutaneous, intramuscular, intravascular or infusion, intra-occular (topical, conjuctival, intra-vitreal, or sub-Tenon), topical, and rectal administration.

The compound may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository. The compound may be in unit dosage forms suitable for single administration of precise dosages. Preferably, dosage forms include a conventional pharmaceutical carrier or excipient and the compound of formula 1 or 2 as an active ingredient. In addition, dosage forms may include other medicinal or pharmaceutical agents, carriers, adjuvants, etc. Preferred formulations are described in U.S. patent application Ser. No. 10/658,801, filed Sep. 10, 2003, and corresponding PCT Publication No. WO 04/024127, the disclosures of which are incorporated herein by reference in their entireties.

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

In preferred embodiments of the dosage forms of the invention, the dosage form is an oral dosage form, more preferably, a tablet or a capsule.

In preferred embodiments of the methods of the invention, the compound of formula 1 or 2 is administered orally, such as, for example, using an oral dosage form as described in U.S. patent application Ser. No. 10/658,801, filed Sep. 10, 2003, and corresponding PCT Publication No. WO 04/024127.

The methods include administering the compound of formula 1 or 2 using an intermittent dosing regimen including a treatment period and a rest period, where one or both of the compounds of formula 1 and 2, or a pharmaceutically acceptable salt, solvate or hydrate thereof, is administered in the treatment period, and administration of the compound is discontinued in the rest period. The specific duration of the treatment period, and the specific dosage administered, are readily determined by one skilled in the art, based on factors well known in the art, such as disease progression and the appearance of toxicities not satisfactorily manageable. Likewise, the duration of the rest period is readily determined by one skilled in the art, based on factors well known in the art, such as the return of symptoms, and the lessening or disappearance of drug-related effects. Further, when the treatment regimen includes a combination therapy, it may be desirable to adjust the duration of the treatment and rest periods to better coordinate with combination therapy treatment and rest periods. It should be appreciated that the “rest period” is a period during which the compound 1 or 2 is not administered; administration of other therapeutic agents can be continued or discontinued in the rest period, as desired.

Preferably, the intermittent dosing regimen includes at least two cycles. One skilled in the art can readily determine the appropriate number of cycles, based on factors well known in the art. It should be appreciated that the specific dose chosen, and the duration of treatment and rest periods, need not be the same from cycle to cycle.

As used herein, “blood/plasma concentration” refers to the total blood and/or plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide and 5-(5-fluoro-2-oxo-1,2-dihydroindol-(3Z)-ylidenemethyl)-2,4-dimethyl-1H-pyrrole-3-carboxylic acid (2-ethylaminoethyl)-amide, including those portions of the total concentration that are protein-bound. It should be appreciated that the concentrations of unbound, available 5-(5-fluoro-2-oxo-1,2-dihydroindol-(3Z)-ylidenemethyl)-2,4-dimethyl-1H-pyrrole-3-carboxylic acid (2-diethylaminoethyl)-amide and 5-(5-fluoro-2-oxo-1,2-dihydroindol-(3Z)-ylidenemethyl)-2,4-dimethyl-1H-pyrrole-3-carboxylic acid (2-ethylaminoethyl)-amide are only a small fraction, typically 5-10%, of the total concentration, but it is this total (bound plus free) that is referred to as “blood/plasma concentration”. The contribution of each of the two compounds to the blood/plasma concentration can be from 0 to 100%; for example, if the composition administered is the metabolite, 5-(5-fluoro-2-oxo-1,2-dihydroindol-(3Z)-ylidenemethyl)-2,4-dimethyl-1H-pyrrole-3-carboxylic acid (2-ethylaminoethyl)-amide, the contribution of the parent compound, 5-(5-fluoro-2-oxo-1,2-dihydroindol-(3Z)-ylidenemethyl)-2,4-dimethyl-1H-pyrrole-3-carboxylic acid (2-diethylaminoethyl)-amide, will be essentially zero. The blood/plasma concentration can be measured by various methods well-known in the art; in the Examples herein, blood/plasma concentration was measured by determining the concentration of the compounds liquid chromatography-tandem mass spectrometry (LC-MS/MS) in plasma.

The minimum blood/plasma concentration, Cₘᵢₙ, is the sum of the 5-(5-fluoro-2-oxo-1,2-dihydroindol-(3Z)-ylidenemethyl)-2,4-dimethyl-1H-pyrrole-3-carboxylic acid (2-diethylaminbethyl)-amide and 5-(5-fluoro-2-oxo-1,2-dihydroindol-(3Z)-ylidenemethyl)-2,4-dimethyl-1H-pyrrole-3-carboxylic acid (2-ethylaminoethyl)-amide concentrations, measured at the end of the dosing period. E.g., when dosing is once daily, Cₘᵢₙ is the blood/plasma concentration measured at 24 hours after dosing, before administration of the next dose. It should be appreciated that the specific compound administered can be the compound of formula 1, or the compound of formula 2, or a combination thereof, or pharmaceutically acceptable salts, hydrates or solvates thereof.

During the rest period, the blood/plasma concentration is the concentration measured at any convenient time.

The examples and preparations provided below further illustrate and exemplify the methods of the present invention. It is to be understood that the scope of the present invention is not limited in any way by the scope of the following examples.

### EXAMPLE 1

Sixty-three patients with metastatic renal cell carcinoma (RCC) were treated with repeat cycles of administration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide (L-malate salt) orally at a starting dose of 50 mg daily (free base equivalent mass) for 4 weeks (treatment period), followed by a 2-week rest period. Eligibility included measurable disease, ECOG performance status of 0 or 1, failure of one prior cytokine-based therapy due to disease progression or toxicity, and adequate cardiac function as defined by normal ejection fraction on MUGA or ECHO. Patients were excluded if they had significant cardiac events within the past 12 months or received more than one prior cytokine regimen.

The median age of patients was 60 years and 58 patients (92%) had prior nephrectomy. Best response by RECIST was confirmed partial in 10 patients (16%), stable in 26 patients (41%), progression in 17 patients (27%) and 4 patients (6%) were not evaluated. An additional 6 patients (10%) had reduction of unidimensional measurement of at least 30% and await confirmation of response status. Of 10 patients who achieved a partial response, 1 patient progressed at 5 months and 9 remain progression free at a median follow-up of 4 months from start of therapy. Twelve patients were taken off study for PD, 8 patients for toxicity, and 4 patients for non-compliance/other. A total of 19 patents had their starting dose adjusted after the first cycle; 12 patients were reduced to 37.5 mg and 5 patients were escalated to 62.5 mg. Preliminary safety data (N=44) indicate the most frequent adverse events include fatigue/asthenia (n=30, 67%), nausea (n=22, 50%), diarrhea (n=20, 46%), stomatitis (n=19, 43%), constipation (n=15, 34%), and dyspepsia (n=13, 30%), mostly grades 1 and 2. Two patients had decreases in left ventricular ejection fraction >20% without clinical symptoms and were taken off the study. Grade 4 laboratory results included anemia (n=1), elevated creatinine kinase (n=1), elevated lipase (n=1), and decreased phosphorus (n=1). Grade 3 events included lymphopenia (n=14, 32%), neutropenia (n=4, 9%), anemia (n=2, 5%), leukocytopenia (n=2, 5%), elevated lipase (n=10, 23%) and elevated amylase (n=4, 9%) without signs of pancreatitis; no patients experienced grade 3 thrombocytopenia.

### EXAMPLE 2

Constitutively activating mutations of the receptor FLT3 are present in approximately 30% of patients with AML and correlate with poor prognosis. In pre-clinical experiments, 5-(5-fluoro-2-oxo-1,2-dihydroindol-(3Z)-ylidenemethyl)-2,4-dimethyl-1H-pyrrole-3-carboxylic acid (2-diethylaminoethyl)-amide exhibits dose-dependent efficacy in both an FLT3-ITD xenograft tumor model and a bone marrow engraftment model. Fifteen patients (M/F 6/9, median age 71, range 54-79, median KPS 80, range 60-100) with primary or secondary AML (any FAB type) relapsed or refractory or not amenable to conventional chemotherapy received escalating doses of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, L-malate salt, starting from 50 mg (free base equivalent) daily. Two dose levels (50 and 75 mg) and 2 schedules (Group A: 4 weeks of treatment followed by a 14 day rest period; and Group B: 4 weeks of treatment followed by a 7 day rest period) were evaluated in 3 cohorts of patients. Flt3 genotyping for ITD and D835 mutations was performed to enable retrospective stratification. Correlative laboratory studies to assess inhibition of target phosphorylation, and modulation of signaling pathways regulating apoptosis and proliferation were performed by Western blot and immunohistochemistry analyses and FL expression and IHC were conducted. Eight patients received 50 mg daily and 2 patients 75 mg daily in group A, while 5 patients received 50 mg daily in group B. Twelve patients completed at least 1 cycle of treatment. Most common toxicities observed were: nausea (33%), fatigue (27%), vomiting (13%) and diarrhea (13%), mainly of grade 1-2. Grade 3 drug related fatigue was observed in 20% of patients. One dose limiting toxicity, fatigue grade 4, was observed at 75 mg. Evidence of clinical activity of short duration by WHO criteria was observed in 7 patients [1 morphologic (less than 5% blasts in bone marrow and absence of blasts in peripheral blood without normalization of neutrophils and thrombocytes counts) and 6 partial responses (PR-reduction of blasts by more than 50% in blood and bone marrow)]. In 2 of these 7 patients a rebound of blasts occurred during the treatment-free (rest) period. Three of the responding patients had mutated FLT3 (ITD or D835). One of the patients with FLT3 ITD mutation (Group B) had a 4-month partial response, and showed inhibition of FLT3 phosphorylation following 5-(5-fluoro-2-oxo-1,2-dihydroindol-(3Z)-ylidenemethyl)-2,4-dimethyl-1H-pyrrole-3-carboxylic acid (2-diethylaminoethyl)-amide administration, followed by a decreased FLT3 protein expression within 8 days, consistent with decreasing blast counts. Similar observations were made for 2 other FLT3 mutant patients analyzed, confirming in vivo target inhibition with the dosing regimens employed. In bone marrow biopsies from the FLT3-ITD patient with the 4-month partial response, decreased expression of KIT, KDR, pSTAT5, pAKT and Ki67 was apparent within 1 cycle of 5-(5-fluoro-2-oxo-1,2-dihydroindol-(3Z)-ylidenemethyl)-2,4-dimethyl-1H-pyrrole-3-carboxylic acid (2-diethylaminoethyl)-amide treatment. Preliminary conclusions: Target inhibition was consistently observed at the 50 mg dose of 5-(5-fluoro-2-oxo-1,2-dihydroindol-(3Z)-ylidenemethyl)-2,4-dimethyl-1H-pyrrole-3-carboxylic acid (2-diethylaminoethyl)-amide, that is tolerable in this advanced AML population. There is preliminary indication of biological activity even though of short duration. The results are summarized in Table 1.

TABLE 1

| | | Schedule | | | |
|---|---|---|---|---|---|
| | Dose | (weeks treatment/ | No. | Best | Reason |
| Pt. No. | (mg daily) | weeks rest) | Cycles | Response | Off-study |
| 1 | 50 | 4/2 | <1 | NE | PD |
| 2 | 50 | 4/2 | 2 | PR | PD |
| 3 | 50 | 4/2 | 1 | Morph. R | PD |
| 4 | 50 | 4/2 | 1 | PD | PD |
| 5 | 50 | 4/2 | <1 | PD | PD |
| 6 | 50 | 4/2 | 1 | PD | PD |
| 7 | 50 | 4/2 | 2 | PR | AE(1) |
| 8 | 50 | 4/2 | 2 | PR | PD |
| 9 | 75 | 4/2 | 1 | PR | AE(2) |
| 10 | 75 | 4/2 | <1 | NE | DLT |
| 11 | 50 | 4/1 | <1 | SD | AE(3) |
| 12 | 50 | 4/1 | 1 | SD | AE(4) |
| 13 | 50 | 4/1 | 3 | PR | AE(5) |
| 15 | 50 | 4/1 | 3 | PR | PD |
| 16 | 50 | 4/1 | 1 | SD | PD |

DLT: Grade 4 fatigue;
NE: not evaluated;
PR: partial response;
SD: stable disease;
PD: Progressive Disease
AE: 1 - acute mypcardial infarction; 2 - cardiac failure; 3 - sepsis; 4 - pneumonia; 5 - cerebral bleeding

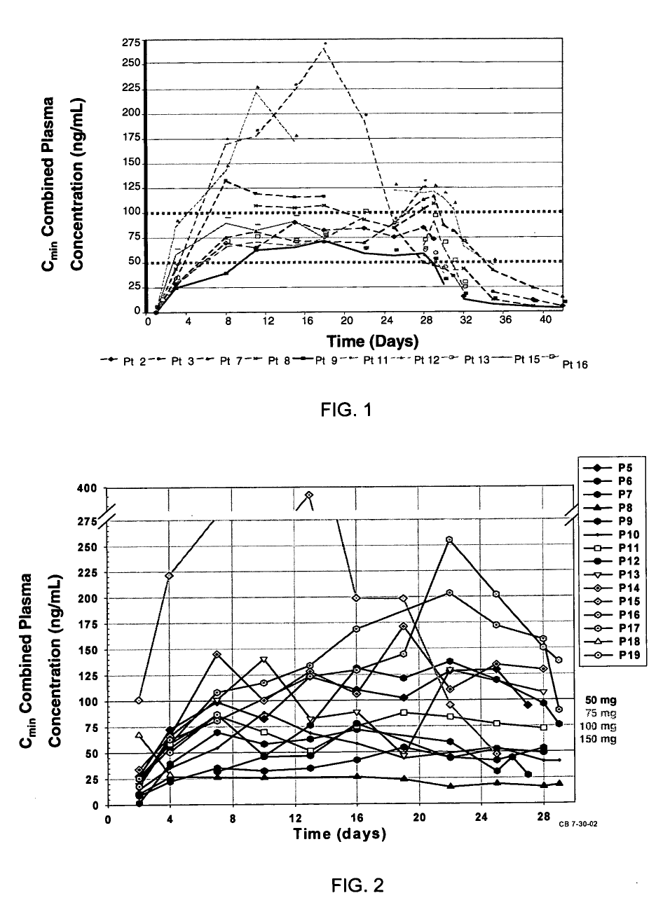

FIG. 1 shows minimum blood/plasma concentrations Cₘᵢₙ for 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide for several patients, taken at various intervals in the cycle.

### EXAMPLE 3

Twenty-eight patients with solid tumors were dosed with 5-(5-fluoro-2-oxo-1,2-dihydroindol-(3Z)-ylidenemethyl)-2,4-dimethyl-1H-pyrrole-3-carboxylic acid (2-diethylaminoethyl)-amide L-malate salt at several dose levels, on a 4 week treatments week rest cycle. The patients were characterized as follows: 15 male, 13 female; age range 33-78 years; median age 55 years; median PS 0; WHO PS, 0 (22 patients), 1 (3 patients), 2 (3 patients). The number of pretreatment regimens ranged from 0 to 9, with a median of 2. The tumors types (number of patients) were: renal cell carcinoma (4); neuroendocrine tumors (4); colorectal cancer (3); non-small cell lung cancer (2); mesotheliomas (2); uterine carcinoma (2); breast cancer (2); pancreas (2); angiosarcoma (2); esophagus (1); undifferentiated carcinoma of nasopharynx (1); parotid adenocarcinoma (1); melanoma (1); gastrointestinal stromal tumor (1). The patients were dosed at starting levels of 59 mg/m², 42 mg/m², 30 mg/m² and 15 mg/m², using 25 mg capsules once daily, except the 15 mg/m² patients who were dosed at 30 mg/m² QOD. Although the trial was started using Body Surface Area; i.e., mg/m², dosing, the data showed no need to adjust (within normal ranges) for weight/height. All other trials were dosed with flat doses (i.e., mg). All dosage amounts are free-base equivalents. The dosing is summarized in Table 2. A plus symbol (+) following the number of cycles indicates the patient was still on study at the date the data were summarized.

TABLE 2

| Patient | Starting Dose | Starting Daily | Number |
|---|---|---|---|
| No. | Level (mg/m²) | Dose (mg) | of Cycles |
| 1 | 15 | 100 | 8+ |
| 2 | 15 | 50 | 1 |
| 3 | 15 | 50 | 2 |
| 4 | 15 | 50 | 2 |
| 5 | 30 | 50 | 6 |
| 6 | 30 | 50 | 1 |
| 7 | 30 | 50 | 1 |
| 8 | 30 | 50 | 1 |
| 9 | 30 | 50 | 2 |
| 10 | 30 | 50 | 2 |
| 11 | 42 | 75 | 4 |
| 12 | 42 | 100 | 5+ |
| 13 | 42 | 75 | 6 |
| 14 | 42 | 75 | 1 |
| 15 | 59 | 150 | 3 |
| 16 | 59 | 100 | 4+ |
| 17 | 59 | 100 | 6+ |
| 18 | 42 | 75 | 1 |
| 19 | 42 | 75 | 3+ |
| 20 | 42 | 75 | 4 |
| 21 | 42 | 75 | 3 |
| 22 | 42 | 75 | 1 |
| 23 | 42 | 75 | 3+ |
| 24 | 42 | 75 | 1 |
| 25 | 42 | 75 | 1+ |
| 26 | 30 | 50 | 1 |
| 27 | 30 | 50 | 2+ |
| 28 | 30 | 50 | 2+ |

FIG. 2 shows minimum blood/plasma concentrations Cₘᵢₙ for 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide for several patients, taken at various intervals in the cycle.

In the 50 mg daily patients, 2 of 9 exhibited dose limiting toxicities at cycle 1: grade 3 edema (1 patient) and grade 4 thrombocytopenia (1 patient). In the 75 mg daily patients, 5 of 11 exhibited dose limiting toxicities at cycle 1: grade 3 asthenia (3 patients), grade 3 hypertension (1 patient) and grade 5 tumor necrosis (1 patient). In the 100 mg daily patients, 2 of 3 exhibited dose limiting toxicities at cycle 1: grade 3 asthenia (1 patient) and grade 3 hypertension (1 patient). The 150 mg daily patient exhibited a dose limiting toxicity of grade 3 asthenia at cycle 1. The remaining patients did not exhibit dose limiting toxicities at cycle 1. Asthenia was the main dose-limiting toxicity at doses greater than or equal to 50 mg, and appeared during cycle 1. Asthenia appeared progressively from grade 1 at week 1 to grade 3-4 at week 4. Grade 3 asthenia was reversible, but required a 2-4 week rest period.

Early evidence of antitumor activity was seen as shown in Table 3.

TABLE 3

| Starting | | Number | Tumor | Tumor | | Tumor |
|---|---|---|---|---|---|---|
| Dosing | | of | Response | Stabilization | | Progres- |
| Schedule | | Patients | (#) | (#), Time | | sion |
| 50 | mg Q2D | 3 | 0 | 0 | | 3 |
| 100 | mg Q2D | 1 | 1 | 0 | | 0 |
| 50 | mg QD | 6 | 0 | 1: 8 | months | 5 |
| 75 | mg QD | 9 | 3 | 2: 4, 5 | months | 0 |
| 100 | mg QD | 3 | 2 | 1: 6 | months | 0 |
| 150 | mg QD | 1 | 0 | 1: 3 | months | 0 |

| | | | | |
|---|---|---|---|---|
| Totals | 23 | 6/23 | 5/23 | 8/23 |
| | evaluable | | | |

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A method of treating abnormal cell growth in a mammal, the method comprising administering to the mammal a therapeutically effective amount of a composition comprising at least one of 5-(5-fluoro-2-oxo-1,2-dihydroindol-(3Z)-ylidenemethyl)-2,4-dimethyl-1H-pyrrole-3-carboxylic acid (2-diethylaminoethyl)-amide or 5-(5-fluoro-2-oxo-1,2-dihydroindol-(3Z)-ylidenemethyl)-2,4-dimethyl-1H-pyrrole-3-carboxylic acid (2-ethylaminoethyl)-amide, or pharmaceutically acceptable salts, solvates or hydrates thereof, for at least one cycle of an intermittent dosing regimen.

2. The method of claim 1, wherein a cycle of the intermittent dosing regimen comprises a treatment period and a rest period, and wherein the intermittent dosing regimen comprises:
(a) administering the composition in the treatment period in an amount sufficient to provide a Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of from 10 to 220 ng/mL; and
(b) discontinuing administration of the composition in the rest period for a duration sufficient to achieve a blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of less than 10 ng/mL.

3. The method of claim 2, wherein the Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide in the treatment period is from 30 to 150 ng/mL.

4. The method of claim 2, wherein the Cₘᵢₙ blood/plasma concentration in the treatment period is from 50 to 100 ng/mL.

5. The method of claim 2, wherein the blood/plasma concentration in the rest period is less than 5 ng/mL.

6. The method of claim 2, wherein the blood/plasma concentration in the rest period is less than 1 ng/mL.

7. The method of claim 1, wherein the therapeutically effective amount is from 10 to 100 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

8. The method of claim 1, wherein the therapeutically effective amount is from 20 to 80 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

9. The method of claim 1, wherein the therapeutically effective amount is from 30 to 75 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

10. The method of claim 1, wherein the therapeutically effective amount is about 50 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

11. The method of claim 1, wherein the composition is administered for at least two cycles of the intermittent dosing regimen.

12. The method of claim 2, wherein the composition is administered at least once per day during the treatment period.

13. The method of claim 2, wherein the composition is administered at least once per two days during the treatment period.

14. The method of claim 2, wherein the treatment period has a duration of at least 7 days.

15. The method of claim 2, wherein the treatment period has a duration of at least 14 days.

16. The method of claim 2, wherein the treatment period has a duration of at least 21 days.

17. The method of claim 2, wherein the treatment period has a duration of at least 28 days.

18. The method of claim 2, wherein the rest period has a duration of at least 3 days.

19. The method of claim 2, wherein the rest period has a duration of at least 5 days.

20. The method of claim 2, wherein the rest period has a duration of at least 7 days.

21. The method of claim 2, wherein the rest period has a duration of at least 10 days.

22. The method of claim 2, wherein the treatment period has a duration of 28 days, and the rest period has a duration of 14 days.

23. The method of claim 1, wherein the abnormal cell growth is cancer.

24. The method of claim 23, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

25. The method of 23, wherein the cancer is selected from gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, and combinations thereof.

26. The method of claim 23, wherein the cancer is renal cell carcinoma.

27. The method of claim 1, wherein the method further comprises co-administering an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, anti-androgens and mixtures thereof.

28. A method of treating cancer in a mammal, the method comprising:
(a) administering to the mammal in a treatment period, a therapeutically effective amount of a composition comprising at least one of 5-(5-fluoro-2-oxo-1,2-dihydroindol-(3Z)-ylidenemethyl)-2,4-dimethyl-1H-pyrrole-3-carboxylic acid (2-diethylaminoethyl)-amide or 5-(5-fluoro-2-oxo-1,2-dihydroindol-(3Z)-ylidenemethyl)-2,4-dimethyl-1H-pyrrole-3-carboxylic acid (2-ethylaminoethyl)-amide, or pharmaceutically acceptable salts, solvates or hydrates thereof, in an amount sufficient to provide a Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of from 10 to 220 ng/mL;
(b) discontinuing administration of the composition in a rest period, for a duration sufficient to achieve a blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide of less than 10 ng/mL; and
(c) repeating steps (a) and (b).

29. The method of claim 28, wherein the Cₘᵢₙ blood/plasma concentration of 5-(5-fluoro-2-oxo-1,2-dihydroindol-(3Z)-ylidenemethyl)-2,4-dimethyl-1H-pyrrole-3-carboxylic acid (2-diethylaminoethyl)-amide plus 5-(5-fluoro-2-oxo-1,2-dihydroindol-(3Z)-ylidenemethyl)-2,4-dimethyl-1H-pyrrole-3-carboxylic acid (2-ethylaminoethyl)-amide in the treatment period is from 30 to 150 ng/mL.

30. The method of claim 28, wherein the Cₘᵢₙ blood/plasma concentration in the treatment period is from 50 to 100 ng/mL.

31. The method of claim 28, wherein the blood/plasma concentration in the rest period is less than 5 ng/mL.

32. The method of claim 28, wherein the blood/plasma concentration in the rest period is less than 1 ng/mL.

33. The method of claim 28, wherein the therapeutically effective amount is from 10 to 100 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

34. The method of claim 28, wherein the therapeutically effective amount is from 20 to 80 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

35. The method of claim 28, wherein the therapeutically effective amount is from 30 to 75 mg per day expressed as free base equivalent mass of 5-(5-fluoro-2-oxo-1,2-dihydroindol-(3Z)-ylidenemethyl)-2,4-dimethyl-1H-pyrrole-3-carboxylic acid (2-diethylaminoethyl)-amide.

36. The method of claim 28, wherein the composition is administered at least once per day during the treatment period.

37. The method of claim 28, wherein the composition is administered at least once per two days during the treatment period.

38. The method of claim 28, wherein the treatment period has a duration of at least 7 days.

39. The method of claim 28, wherein the treatment period has a duration of at least 14 days.

40. The method of claim 28, wherein the treatment period has a duration of at least 21 days.

41. The method of claim 28, wherein the treatment period has a duration of at least 28 days.

42. The method of claim 28, wherein the rest period has a duration of at least 3 days.

43. The method of claim 28, wherein the rest period has a duration of at least 5 days.

44. The method of claim 28, wherein the rest period has a duration of at least 7 days.

45. The method of claim 28, wherein the rest period has a duration of at least 10 days.

46. The method of claim 28, wherein the cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, and combinations thereof.

47. The method of 28, wherein the cancer is selected from gastrointestinal stromal tumors, renal cell carcinoma, breast cancer, colorectal cancer, non-small cell lung cancer, neuroendocrine tumors, thyroid cancer, small cell lung cancer, mastocytosis, glioma, sarcoma, acute myeloid leukemia, prostate cancer, lymphoma, and combinations thereof.

48. The method of claim 28, wherein the cancer is renal cell carcinoma.

49. The method of claim 28, wherein the method further comprises co-administering an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, anti-androgens and mixtures thereof.

50. A method of treating renal cell carcinoma in a patient, the method comprising:
(a) administering to the patient once daily in a treatment period of at least 2 weeks, a composition comprising about 50 mg free base equivalent of 5-(5-fluoro-2-oxo-1,2-dihydroindol-(3Z)-ylidenemethyl)-2,4-dimethyl-1H-pyrrole-3-carboxylic acid (2-diethylaminoethyl)-amide malate salt;
(b) discontinuing administration of the composition in a rest period of at least one week; and
(c) repeating steps (a) and (b).

51. The method of claim 50, wherein the treatment period is about 4 weeks, and the rest period is about 1 week.

52. The method of claim 50, wherein the treatment period is about 4 weeks, and the rest period is about 2 weeks.
